(19) United States
(12) Patent Application Publication
(10) Pub. No.: US 2009/0297499 A1
(43) Pub. Date: Dec. 3, 2009

(54) USE OF CHARCOAL FOR TREATING INFLAMMATORY CONDITIONS

(75) Inventors: Brian M. Foxwell, London (GB); Percy Sumariwalla, London (GB); Paul Kaye, York (GB); Kevin Tracey, New York, NY (US); Kenneth Kenigsberg, Manhasset, NY (US); Luis Ulloa, Manhasset, NY (US)

Correspondence Address: BROOKS KUSHMAN P.C., 1000 TOWN CENTER, TWENTY-SECOND FLOOR, SOUTHFIELD, MI, 48075, US

(73) Assignee: IMPERIAL COLLEGE INNOVATIONS LIMITED, London (GB)

(21) Appl. No.: 11/997,844

(22) PCT Filed: Aug. 4, 2006
(86) PCT No.: PCT/GB06/02908

§ 371 Date: Dec. 11, 2008

(30) Foreign Application Priority Data
Aug. 4, 2005 (GB) 0516069.2

(51) Int. Cl. A61K 33/44; A61P 29/00

(52) U.S. Cl. 424/125

(57) ABSTRACT

The invention relates to the use of charcoal in the manufacture of an oral composition for the treatment of an inflammatory condition other than an inflammatory bowel disease and other than interstitial or other inflammation within the kidney.

The present invention relates to use of charcoal in the manufacture of an oral composition for the treatment of an inflammatory condition other than an inflammatory bowel disease and other than intestinal or other inflammation within the kidney. The present invention also relates to a pharmaceutical composition comprising charcoal in combination with a further anti-inflammatory agent. The present invention further relates to a pharmaceutical composition comprising charcoal in combination with a further anti-inflammatory agent for the treatment of an inflammatory condition and also to a method of treating an inflammatory condition, other than an inflammatory bowel disease and other than intestinal or other inflammation within the kidney, comprising the oral administration of charcoal.

Inflammation is a protective response by the immune system to tissue damage and infection. However, the inflammatory response, in some circumstances, can damage the body. In the acute phase, inflammation is characterised by pain, heat, redness, swelling and loss of function. There are a wide range of inflammatory conditions which affect millions of people worldwide. A significant inflammatory condition is rheumatoid arthritis. Rheumatoid arthritis affects 0.5-1% of the human population. This disease is characterised by joint inflammation and leads to progressive debilitation in joint function which results in pain, disability, loss of man power and shorter life expectancy. Multiple sclerosis, lupus, atherosclerosis and cardiovascular disease are also significant inflammatory conditions. The varied symptoms of severe malaria, which includes cerebral malaria largely reflect the consequences of excessive production, in the body, of inflammatory pathway components. Infection with Plasmodium falciparum (an infectious cause of malaria) causes 4-6 million cases of life-threatening severe malaria and over 1 million childhood deaths annually in Africa. A means to reduce/control the symptoms of malaria is hugely desirable. In addition, recent research has indicated that cancer may have an important inflammatory component.

Current treatments for inflammatory conditions have a number of disadvantages, including expense and/or severe side effects. At present, steroids such as dexamethasone, are widely used in the treatment of inflammatory conditions. While treatment with steroids can be effective, there are a number of serious side effects. These side effects include hypertension, growth deficiencies in younger patients, osteoporosis, cataracts, psychosis, elevated blood sugar, glaucoma, etc. In addition, long-term use of steroids can lead to resistance in some patients.

New and alternative treatment currently used for inflammatory conditions are based on biologicals such as antibodies and soluble receptors. The most widely used of these is based on blocking TNF function with neutralizing antibodies or soluble receptors. This type of anti-TNF therapy has been successful in the treatment of a number of diseases, with a substantial proportion of patients (approximately a third to a quarter) showing significant clinical benefit. However, it is extremely expensive and this places a heavy financial burden either on the patient or the healthcare system or both. Some patients in the developed world and the majority in the developing world are not able to afford this treatment. In addition, possible side effects of anti-TNF therapy include anaphylaxis, cytopenia and increased susceptibility to infection. Currently it is not possible to take anti-TNF drugs orally, which is a disadvantage.

Another class of drugs are the disease modifying anti-rheumatic drugs (DMARDS). An example of these is methotrexate, an anti-metabolite drug, which is widely used for the treatment of rheumatoid arthritis, psoriatic arthritis and psoriasis. Methotrexate has been successful in the treatment of these diseases, but can cause substantial side effects, such as severe skin reaction, infections such as pneumonia, severe damage to liver, kidneys, lungs and gastrointestinal tract.

A number of DMARD pharmaceutical agents containing gold are also used in the treatment of inflammatory conditions, particularly rheumatoid arthritis. Examples of such agents include gold sodium thiomalate and auranofin. Potential side effects from being treated with anti-inflammatory gold agents are oral ulcers, altered taste, serious skin rashes, renal problems, inflammation of the intestines (enterocolitis), liver injury and lung disease. Furthermore, resistance to gold has been known to develop in patients.

A further class of drugs are the non-steroidal anti-inflammatory drugs (NSAID's). These are used to alleviate symptoms and includes the Cox 2 inhibitors “VIOXX”® (a registered trademark of Merck & Co., Inc) and “CELEBREX”®, (a registered trademark of G.D. Searle & Co).

As a result of lack of efficacy, development of resistance, unacceptable side-effects and expense of existing treatments, it is hugely desirable to find an alternative treatment for inflammatory conditions.

Charcoal is well known for use in emergency treatment for specific types of poisoning and blood overdoses. Charcoal is also used to treat digestive complaints such as intestinal gas (flatulence), diarrhoea, and stomach ulcer pain.

There has been some investigation of the use of charcoal in the treatment of inflammatory bowel disease. U.S. Pat. No. 5,554,370 describes a method of treating a patient suffering from inflammatory bowel disease by oral administration of spherical activated charcoal. In the use described in U.S. Pat. No. 5,554,370 there is direct contact between the tissue to be treated and the charcoal. The charcoal has a local effect.

There has been some investigation of the use of charcoal to reduce disorders within the kidney, including interstitial inflammation. Aoyama 1., Stimokata K., and Niwa T., Neptron, 2002, 92:635-651 describe the use of charcoal to delay the progression of renal failure. In the use described in this document (in a rat model only), the charcoal acts by removing uremic toxins, such as indoxyl sulphate in order to ameliorate the development of interstitial inflammation. Indoxyl sulphate is a metabolic product of indole and indole can be produced by gut flora. The literature suggests that the mechanism by which charcoal reduces inflammation in the kidney is the absorption of indole in the gut before it is metabolised to indoxyl sulphate and absorbed by the body.

Charcoal has been used in various hemoperfusion treatments. These types of treatments involve removing blood from the body and circulating the blood past charcoal to remove toxins, drugs, etc for the treatment of sepsis or septic shock. Hemoperfusion treatment using charcoal has also been investigated for rheumatoid arthritis (Martynov et al., 1992 Ter Arkh. 64(7): 103-7). There is no suggestion in this document that charcoal could be effective for the treatment of rheumatoid arthritis, if taken orally. In fact, if the authors of this paper had any reason to believe that orally administered charcoal could be used to treat rheumatoid arthritis, then hemoperfusion using charcoal would not have been pursued. This is because hemoperfusion is considerably more traumatic for a patient than the oral administration of a medicament. Furthermore, hemoperfusion is expensive, as equipment and qualified medical personnel are required. The teaching of this Martynov et al paper is that the factors affecting rheumatoid arthritis are present in the blood and that the blood must be contacted directly with the charcoal to have the desired effect.

A treatment for malaria is a world-wide aim. Although the pathophysiologic basis of severe malaria is yet to be fully defined, arguments have been put forward for the role of pro-inflammatory cytokines in the disease. Failure to break the vicious cycle of metabolic changes induced by excess cytokine production contributes significantly to the high mortality rates observed, in spite of increasingly effective anti-malarial drugs. Attempts to improve survival by targeting individual cytokines, notably TNF, have been largely unsuccessful.

Severe sepsis, the third leading cause of death in developed countries, is also mediated by cytokine over-expression, but anti-TNF therapies, and other strategies to target specific cytokines have yet to be proven effective in clinical trials.

The first aspect of the present invention provides the use of charcoal in the manufacture of an oral composition for the treatment of an inflammatory condition other than an inflammatory bowel disease and other than interstitial or other inflammation within the kidney. By inflammatory bowel disease is meant a general term for intestinal inflammation. Such a composition is preferably a medicament.

In particular, charcoal is useful for treating inflammatory conditions such as autoimmune inflammatory conditions, particularly rheumatoid arthritis (including juvenile rheumatoid arthritis), psoriatic arthritis, cardiovascular disease, glaucoma, sarcoidosis, endometriosis, multiple sclerosis, ankylosing spondylitis, atherosclerosis, lupus, psoriasis, glomerulonephritis; malarial inflammatory conditions, particularly malaria (which may be severe malaria) including cerebral malaria; inflammation associated with cancer; lung associated inflammatory diseases particularly severe acute respiratory syndrome (SARS), influenza, in particular influenza induced inflammation, chronic asthma and chronic obstructive pulmonary disease (COPD); infection associated inflammation, including malaria, influenza, as well as other infections such as bacterial and viral infections, sepsis, endotoxemia; and/or injury-associated inflammation (as exemplified by air pouch model(s)) including burning, bruising, swelling, breakages and post surgery-associated inflammation.

According to the present invention, the charcoal can be used to treat any one inflammatory condition or a combination of inflammatory conditions at the same or different time(s).

There is no limitation as to the type of charcoal to be used. Preferably the charcoal is activated charcoal. The activated charcoal is preferably of clinical grade.

Typically, activated charcoal is produced by heating charcoal with steam to approximately 1000° C. in the absence of oxygen. This treatment removes residual non-carbon elements and produces a porous internal microstructure having an extremely high surface area. Activated charcoal typically has particle sizes of 0.05 to 2 mm, a specific surface area of 500 to 2 000 m²/g and a specific pore volume of 0.2 to 2.0 ml/g determined in the range of a pore radius of not more than 80 Å.

Charcoal has an inert and harmless structure and can be taken orally with no known side effects. In addition, charcoal does not suppress the immune system of a subject, and therefore does not make the subject more susceptible to infection.

In the present invention, the charcoal-containing medicament is administered orally. The effect of the oral administration is understood to be systemic. As a result, the charcoal is effective in treating inflammatory conditions which afflict parts of the body that do not come into direct contact with the charcoal. This is particularly surprising in view of the teachings of the prior art.

A dose of charcoal is preferably between 0.25 g and 100 g. One dose may be effective or more than one may be necessary. The dose regime may be once daily, more than once daily, weekly or monthly. The content of charcoal in the pharmaceutical compositions may be anywhere between 1 to 100 wt. % of the composition.

A particular advantage of the present invention is that charcoal is extremely cheap in comparison to most of the treatments currently available for the treatment of inflammatory conditions and appear to have no known unacceptable side effects.

Given the urgent need for treatment of life-threatening diseases such as severe malaria, the application of charcoal is particularly useful as it can be rapidly available for clinical use.

The charcoal-containing medicament may be used in combination with a further anti-inflammatory agent. Administration of the charcoal and other anti-inflammatory agent can be simultaneous, separate and/or sequential. The charcoal, in combination with another pharmaceutical agent, can act additively or synergistically.

The other anti-inflammatory agent may be termed a non-steroidal anti-inflammatory agent (NSAID), a disease modifying anti-rheumatic drug (DMARD), a biological agent (biologicals), a steroid, an immunosuppressive agent, a salicylate and/or a microbicidal agent. Non-steroidal anti-inflammatory agents include anti-metabolite agents (including methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-B cell reagents (retoximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptemers, antisense oligonucleotides or omega 3 fatty acids. Steroids include cortisone, prednisolone or dexamethasone. Immunosuppresive agents include cylcosporin, FK506, rapamycin, mycophenolic acid. Salicylates include aspirin, sodium salicylate, choline salicylate and magnesium salicylate. Microbicidal agents include quinine and chloroquine.

The further anti-inflammatory agent is administered by any appropriate route, for example oral (including buccal or sublingual), topical (including buccal, sublingual or transdermal), or parenteral (including subcutaneous, intramuscular, intravenous or intradermal) route. Where the further anti-inflammatory agent is administered orally, it may be administered as part of the same composition as the charcoal.

The second aspect of the invention is a pharmaceutical composition comprising charcoal in combination with a further anti-inflammatory agent. Preferably, the composition of the second aspect is an oral composition.

The third aspect of the invention is a pharmaceutical composition comprising charcoal in combination with a further anti-inflammatory agent for the treatment of an inflammatory condition. The inflammatory condition may be other than an inflammatory bowel disease and other than interstitial or other inflammation within the kidney. The composition according to the third aspect of the invention is preferably an oral composition.

The fourth aspect of the invention is a method of treating an inflammatory condition other than an inflammatory bowel disease and other than interstitial or other inflammation within the kidney comprising the oral administration of charcoal. In the fourth aspect of the invention, the method is carried out on a subject in need of treatment or a subject whom has been identified as having an increased susceptibility (or disposition) to suffering from one or more of the inflammatory conditions according to the invention. In the method of the fourth aspect, it may involve one or more steps to either determine the subject's susceptibility to an inflammatory condition of the invention or it may involve one or more steps to monitor the subject after the treatment has been carried out. The subject's susceptibility may involve an invasive or non-invasive diagnostic test, including requesting information from the patient as to their family history health in relation to inflammatory conditions. Monitoring of the subject after treatment may involve invasive or non-invasive testing, including requesting information from the subject or testing as to one or more of the following; pain levels, comfort levels, mobility of joints, ease of breathing while resting or while exercising, body temperature levels, ability to exercise, vomiting levels etc.

The preferred embodiments, as described for the first aspect of the invention, are the same for all aspects of the invention.

Compositions in accordance with the invention may be supplied as part of a sterile, pharmaceutical composition which will normally include a pharmaceutically acceptable carrier. This pharmaceutical composition may be in any suitable form. It may be provided in unit dosage form, will generally be provided in a sealed container and may be provided as part of a kit. Such a kit would normally (although not necessarily) include instructions for use. It may include a plurality of said unit dosage forms.

The oral pharmaceutical compositions may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions oils (e.g. vegetable oils) may be used to provide oil-in-water or water in oil suspensions.

The pharmaceutical compositions may contain preserving agents, solubilising agents, stabilising agents, wetting agents, emulsifiers, sweeteners, colourants, odourants, salts, buffers, coating agents or antioxidants. They may also contain further therapeutically active agents in addition to the anti-inflammatory agents of the present invention.

Dosages of the substances of the present invention can vary between wide limits, depending upon the condition to be treated, the health of the individual to be treated, etc. and a physician may determine appropriate dosages to be used. The dosage may be repeated as often as appropriate.

The compositions and uses described in this application are envisaged to have human, animal and veterinary applications. They are preferably applicable to mammals, in particular humans, but are also applicable for use in production animals, in particular sheep, cows, pigs, chickens and goats, as well as companion animals, in particular cats and dogs and sporting animals, such as horses.

In the present invention, the term “treatment” includes prophylactic treatment (i.e. prevention) and therapeutic treatment. In most circumstances, prevention of an inflammatory condition is unlikely to be carried out. Usually, it is only when the presence of an inflammatory disease is diagnosed in a subject that prevention means are applied. However, prophylactic treatment may be appropriate if there is i) a known family history of significant inflammatory conditions or if tests (e.g. genetic tests) identify that an individual has a predisposition to one or more inflammatory conditions of the invention or ii) an increased risk of suffering from one or more inflammatory conditions, such as an increased risk of contracting malaria.

The present invention is described with references to the drawings, in which:

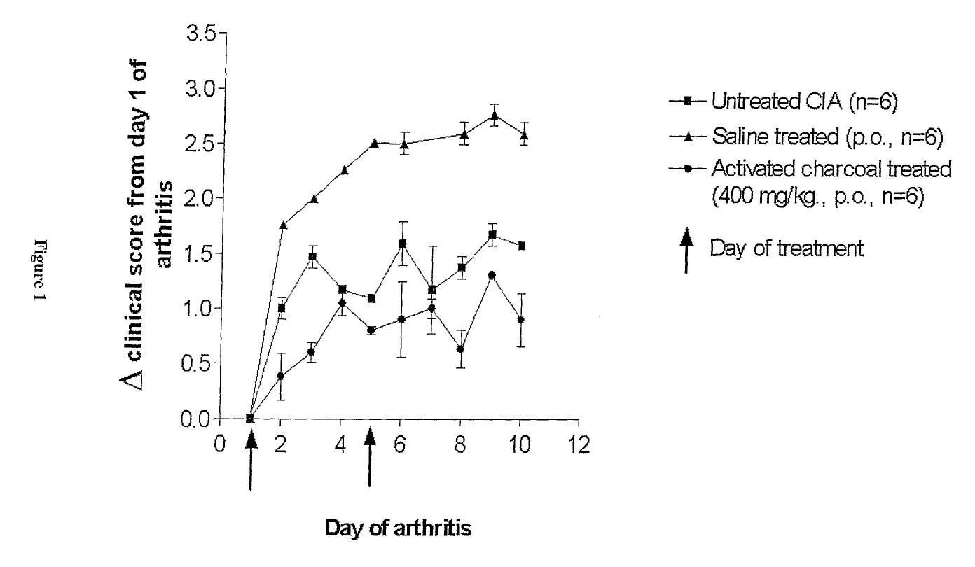

FIG. 1 illustrates the clinical score of mice with collagen induced arthritis treated with activated charcoal compared to the controls of untreated mice with collagen induced arthritis and saline treated mice with collagen induced arthritis from experiment 1 of Example 1.

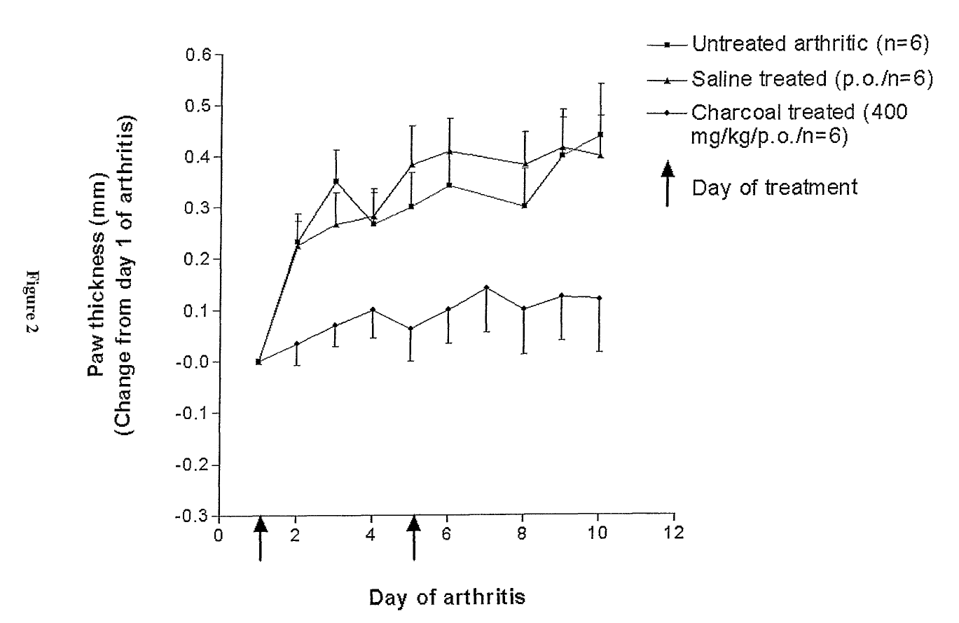

FIG. 2 illustrates the paw thickness (mm) of mice with collagen induced arthritis treated with activated charcoal compared to the controls of untreated mice with collagen induced arthritis and saline treated mice with collagen induced arthritis from experiment 1 of Example 1.

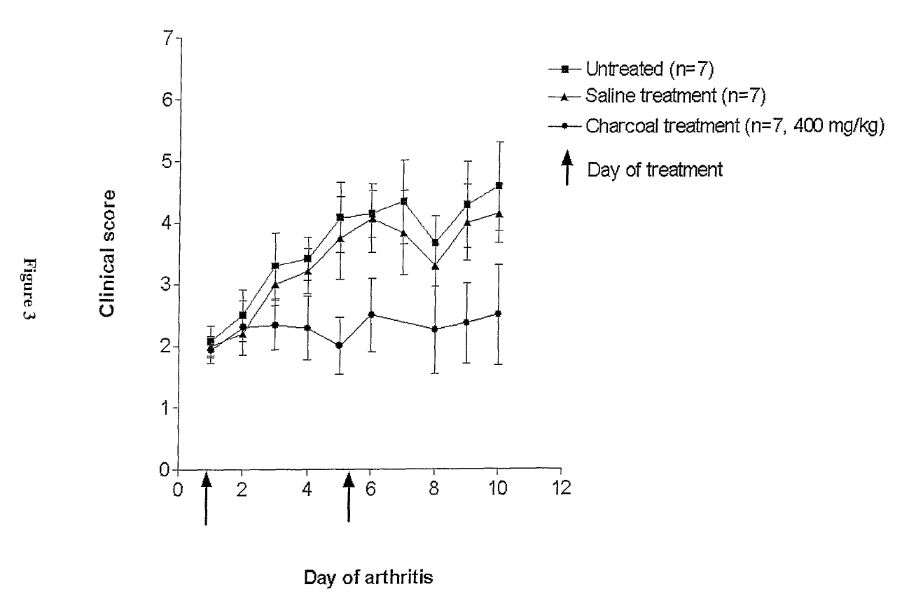

FIG. 3 illustrates the clinical score of mice with collagen induced arthritis treated with activated charcoal compared to the controls of untreated mice with collagen induced arthritis and saline treated mice with collagen induced arthritis from experiment 2 of Example 1.

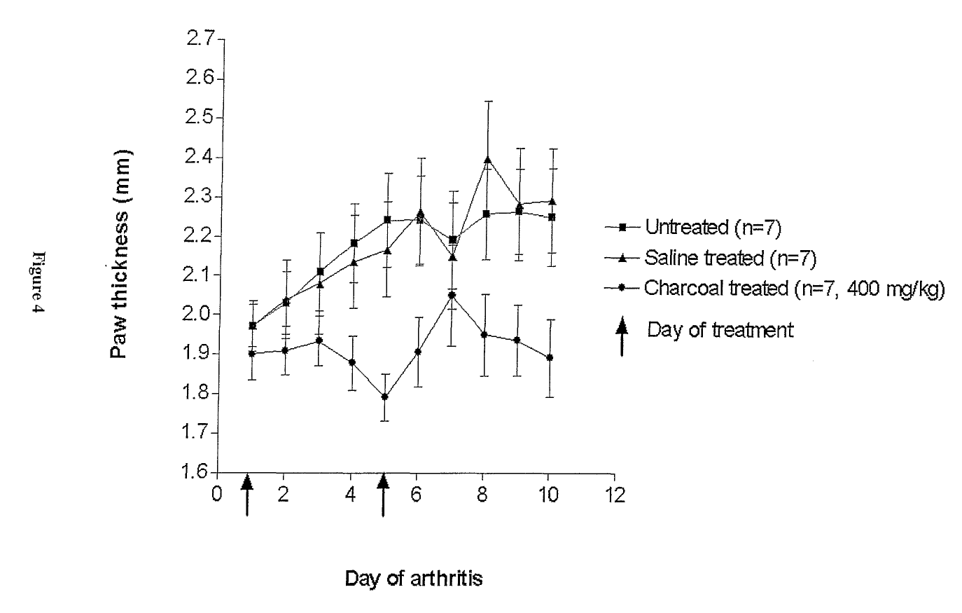

FIG. 4 illustrates the paw thickness (mm) of mice with collagen induced arthritis treated with activated charcoal compared to the controls of untreated mice with collagen induced arthritis and saline treated mice with collagen induced arthritis from experiment 2 of Example 1.

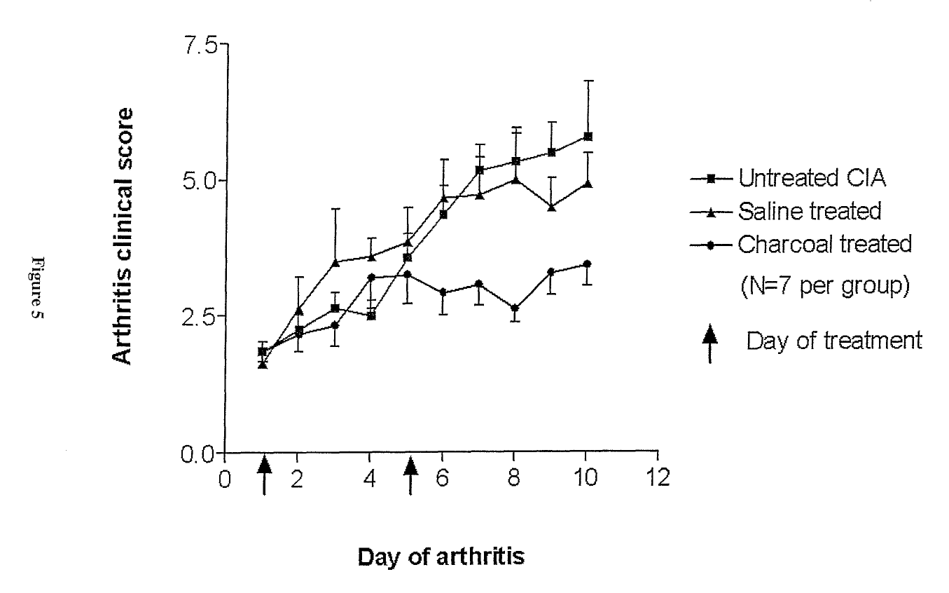

FIG. 5 illustrates the clinical score of mice with collagen induced arthritis treated with activated charcoal compared to the controls of untreated mice with collagen induced arthritis and saline treated mice with collagen induced arthritis from experiment 3 of Example 1.

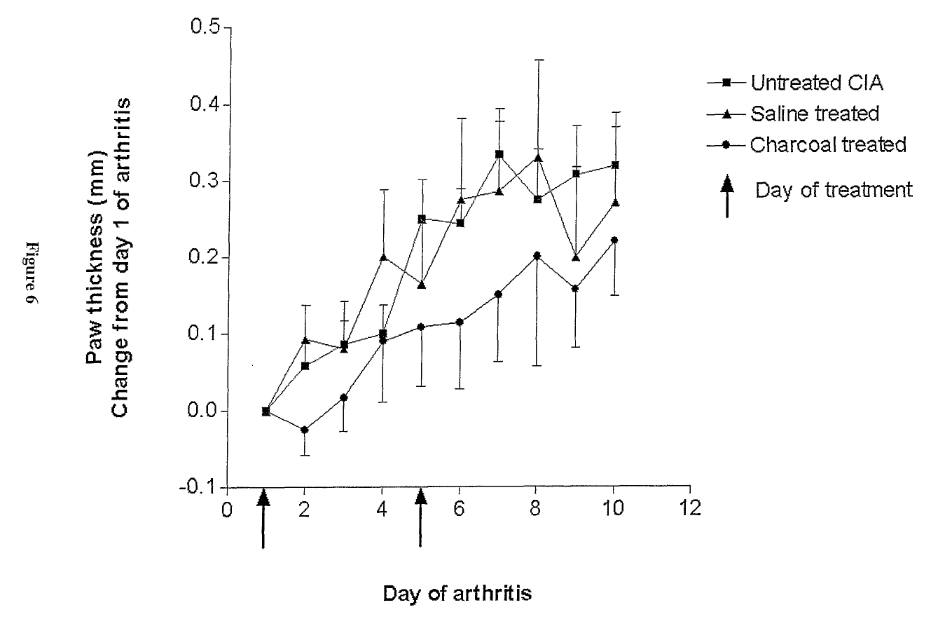

FIG. 6 illustrates the paw thickness (mm) of mice with collagen induced arthritis treated with activated charcoal compared to the controls of untreated mice with collagen induced arthritis and saline treated mice with collagen induced arthritis from experiment 3 of Example 1.

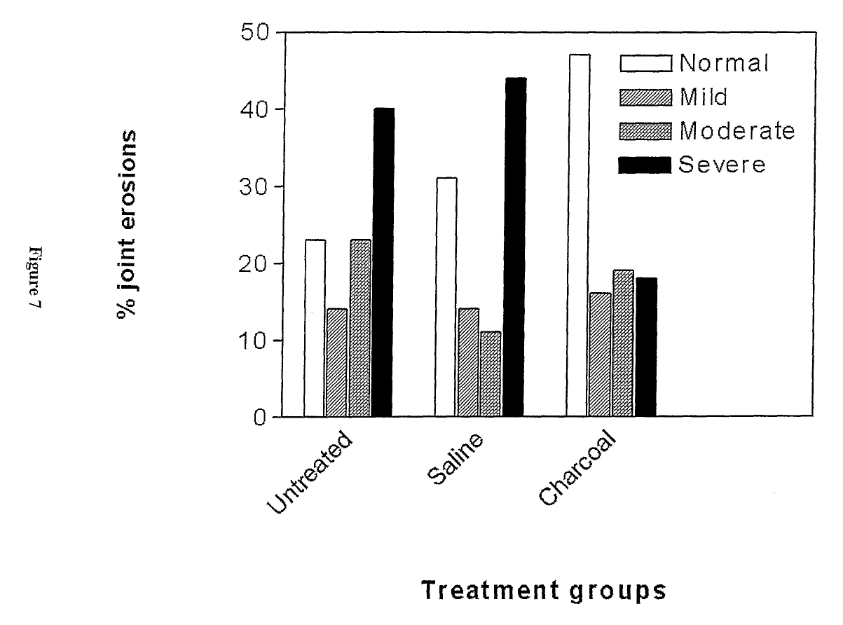

FIG. 7 illustrates the composite histological profile of all joints from mice with collagen induced arthritis treated with activated charcoal compared to the controls of untreated mice with collagen induced arthritis and saline treated mice with collagen induced arthritis from experiments 1-3 of Example 1.

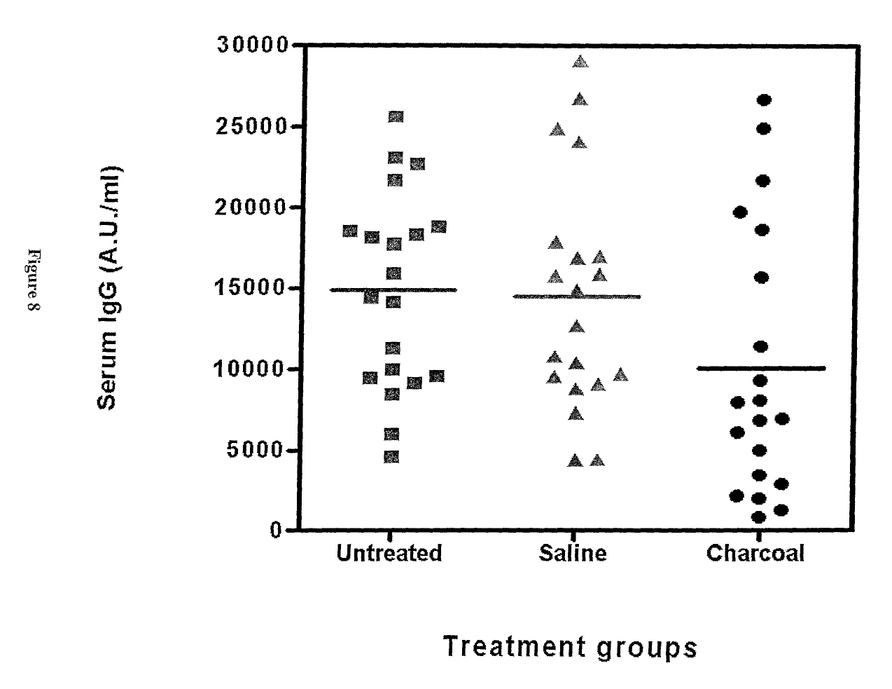

FIG. 8 illustrates the serum anti bovine CII IgG (total) levels of all mice with collagen induced arthritis treated with activated charcoal compared to the controls of untreated mice with collagen induced arthritis and saline treated mice with collagen induced arthritis from experiments 1-3 of Example 1.

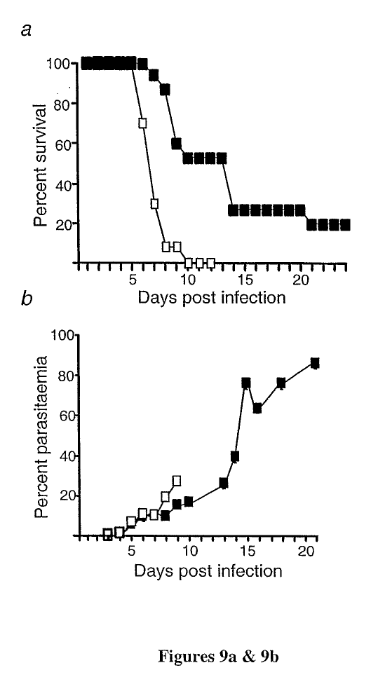

FIG. 9 illustrates the use of activated charcoal to protect mice against cerebral malaria (cm). In FIG. 9a C57BL/6 mice were infected with P. berghei ANKA and either left untreated (□) or treated with activated charcoal on days 3 and 5 (▪). Mice were monitored daily for the development of CM and for survival. Results represent pooled data derived from 3 independent experiments (n=13 per group). Differences in survival were highly significant (X²=19.18; P<<0.0001). In FIG. 9b Parasitemia in control (□) and charcoal-treated (▪) mice (Example 2).

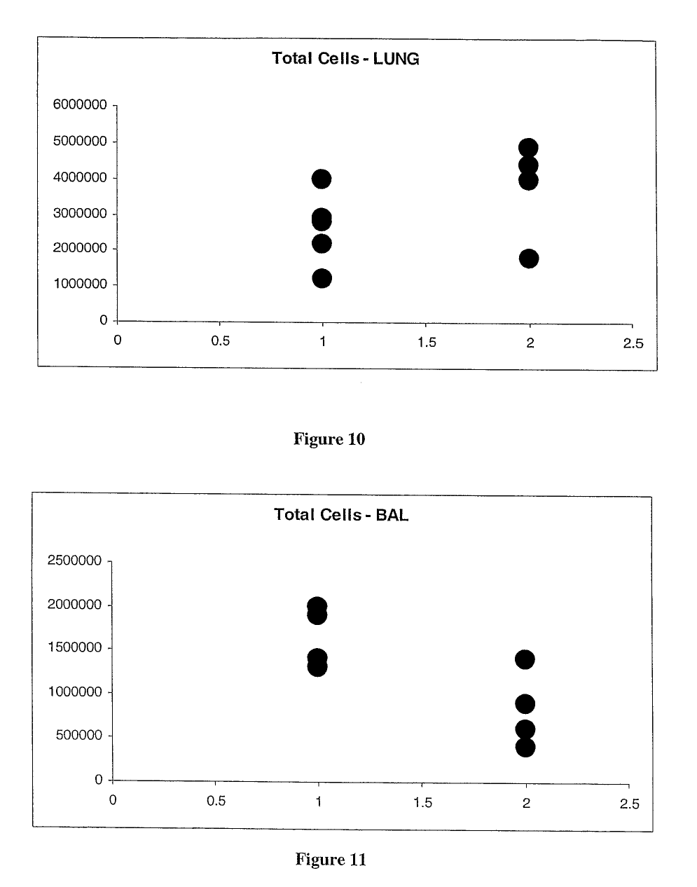

FIG. 10 illustrates the total number of cells in the lungs of the influenza infected mice. The number 1 on the X axis represents the saline treated mice and the number 2 on the X axis represents the charcoal treated mice. The Y axis represents the number of cells (Example 3).

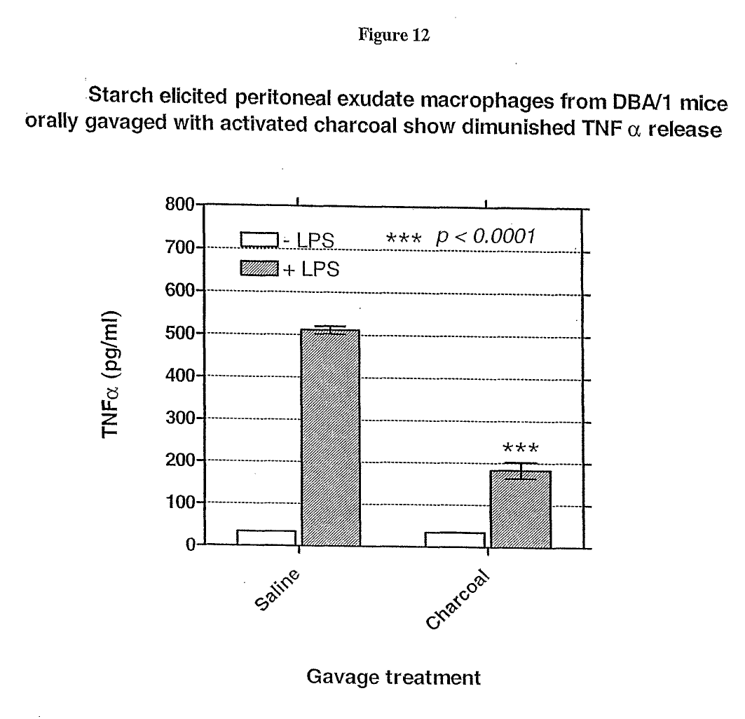

FIG. 11 illustrates the total number of cells in the bronchoalveolar lavage, which represents the total number of cells in the airways of the lungs of the influenza infected mice. The number 1 on the X axis represents the saline treated mice and the number 2 on the X axis represents the charcoal treated mice. The Y axis represents the number of cells (Example 3).

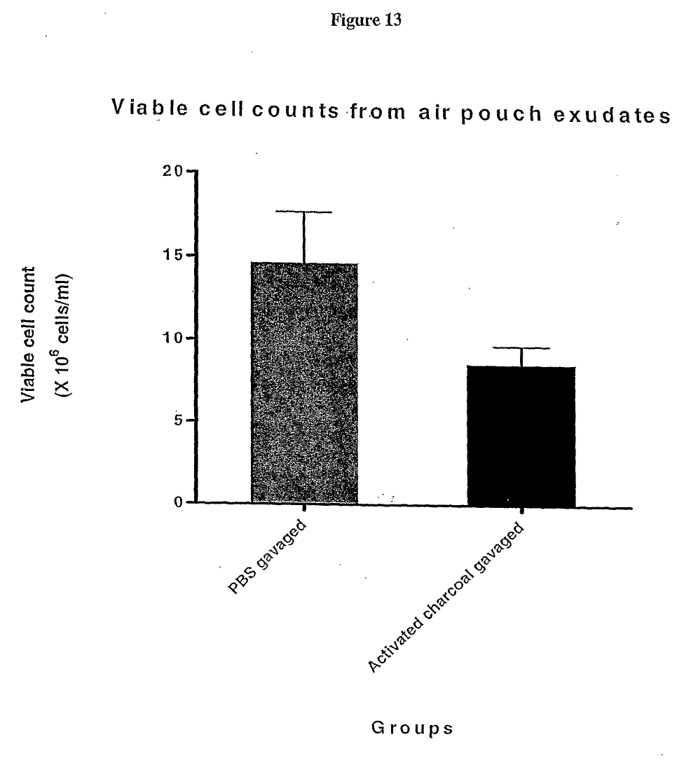

FIG. 12 illustrates the amount of TNFα release in starch elicited peritoneal exudates macrophages from mice orally gavaged with activated charcoal or with saline (Example 4).

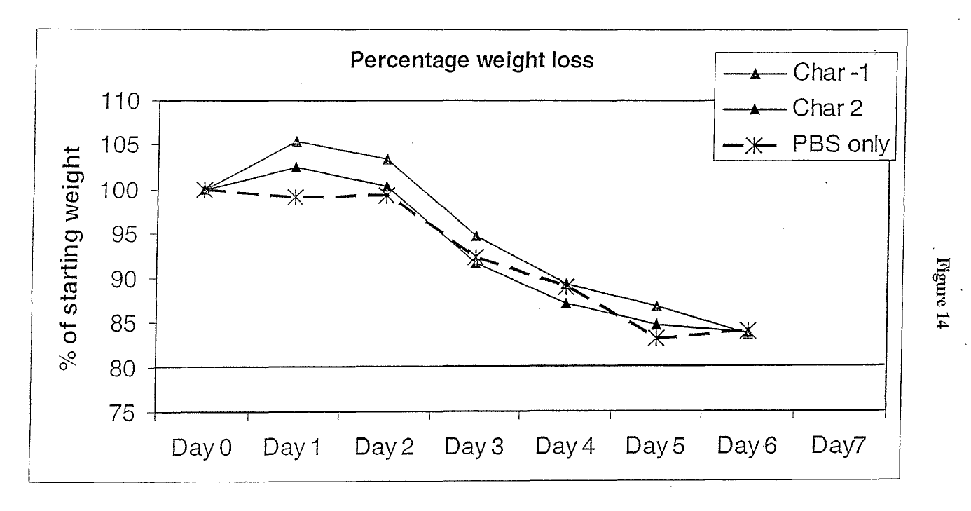

FIG. 13 illustrates the viable cell count from air pouch exudates, in an air pouch model inflammation, of mice orally gavaged with either saline or charcoal (Example 5).

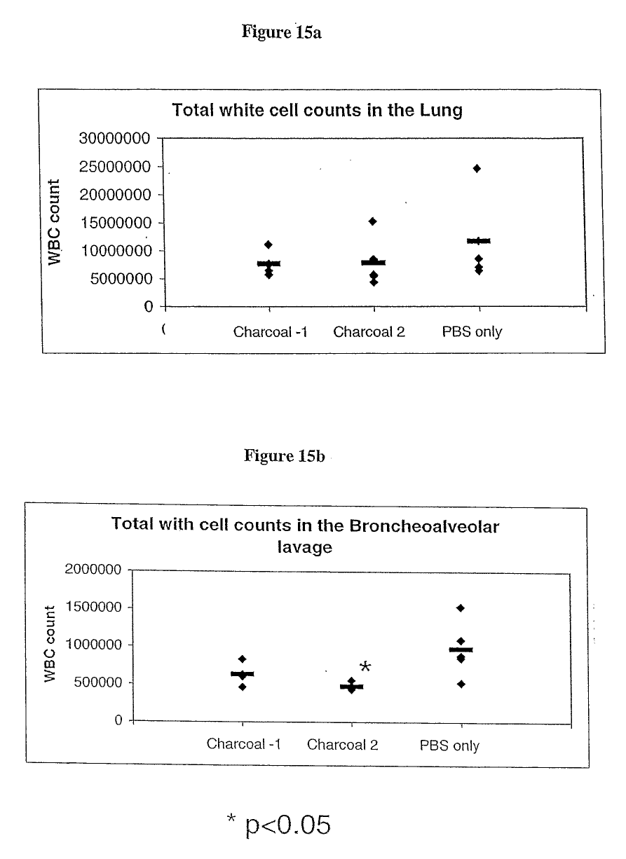

FIG. 14 illustrates percentage weight loss of mice over time (Example 6).

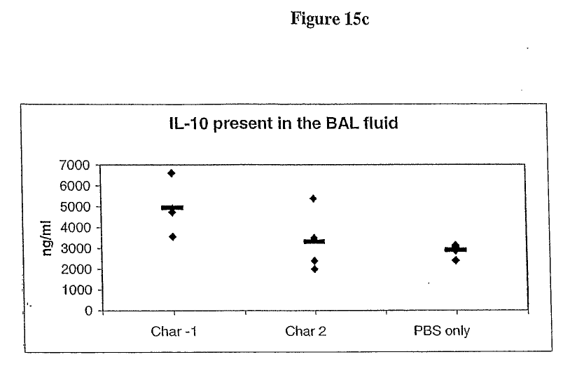

FIGS. 15a, b and c illustrate either white blood counts or amount of IL-10 in mice treated with charcoal compared to the control mice (Example 6).

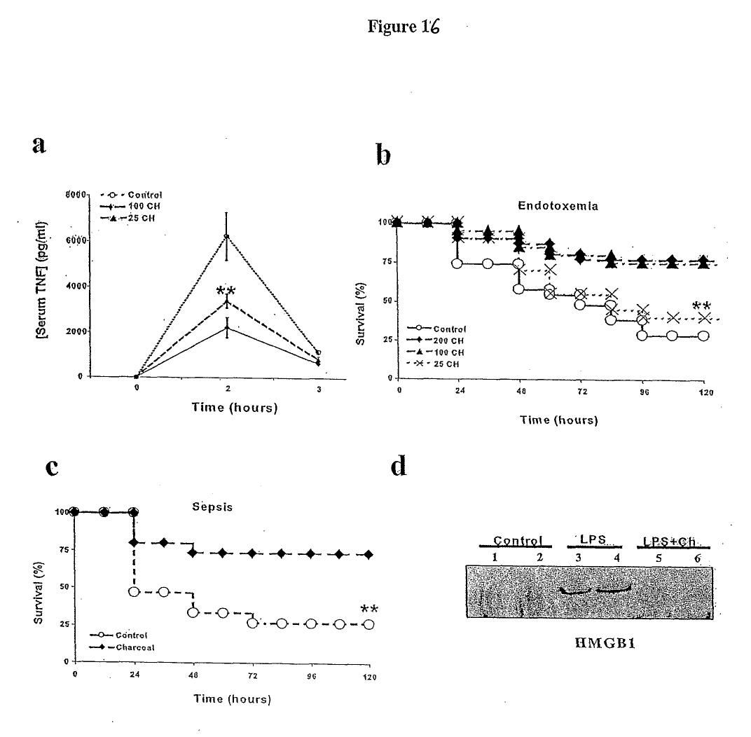

FIG. 16a illustrates the serum TNF levels of mice in different experimental or control groups, over time (Example 7).

FIG. 16b illustrates percent survival of mice in different experimental or control groups, over time (Example 7).

FIG. 16c illustrates percentage survival of mice in different experimental or control groups, over time (Example 7).

FIG. 16d illustrates inhibition of HMGB1 levels by activated charcoal (Example 7).

The present invention is described with reference to the following non-limiting examples:

### EXAMPLE 1

Treatment of rheumatoid arthritis with charcoal was investigated using the murine collagen-induced arthritis (CIA) model. This model is widely used as an experimental model for rheumatoid arthritis.

Six DBA/1 male mice (experiment 1) or seven DBA/1 mice (experiments 2 and 3) at 10 weeks old were injected with a single injection of 100-200 μg of bovine type II collagen and Freund's complete adjuvant (FCA). DBA1 mice are susceptible to the induction of arthritis.

The paws of the mice were examined for the clinical signs of arthritis characterised by oedema and erythema. Once the clinical signs had been observed the mice were orally administered with 400 mg/kg activated charcoal one day and five days after the onset of the clinical signs of arthritis. The mice were monitored for clinical scores and paw thickness (mm).

After ten days from the onset of the clinical signs of arthritis, the mice were culled and the paws of the mice from experiment 1 were examined for histology and the blood of the mice from experiments 1-3 was examined for serology.

The results indicated a reduction in the clinical score and reduced paw thickness (mm) in response to the activated charcoal treatment when compared to the untreated and saline treated mice in all three experiments (FIGS. 1-6).

The histological profile of the mice from experiment 1 demonstrated that the mice treated with activated charcoal suffered less than half the percentage of severe joint erosions that untreated mice and saline treated mice suffered (FIG. 7). This indicates that charcoal treated mice exhibit an increased degree of protection from inflammatory damage.

In addition, the serum anti bovine CII IgG (total) levels in activated charcoal treated mice from experiments 1-3 were significantly lower (p<0.05 Mann-Whitney U-test) than saline treated mice (FIG. 8).

### EXAMPLE 2

We used the model of cerebral malaria (CM) caused by Plasmodium berghei ANKA infection in C57BL/6 mice. This is a well-accepted model for many aspects of human disease; pro-inflammatory cytokines are abundant; mice develop central nervous system (CNS) lactic acidosis, increased blood-brain barrier permeability, paralysis, seizures and death; and there are similarities in brain histopathology. 6 to 8-week old C57BL/6 mice (20-25 g) were purchased from Charles River Laboratories and maintained under barrier conditions with free access to water and diet. Mice were infected by intravenous injection of 10⁴ parasitized RBCs obtained from infected C57BL/6 mice, and were monitored daily for neurological signs of CM, including convulsions, ataxia and paralysis. Parasitaemias were determined from stained blood films. Actidose-Aqua activated charcoal (0.2 g charcoal/ml) was obtained from Paddock laboratories, Inc. (Cat# NDC0574-0121-04), and mice were dosed on days 3 and 5 post infection with 130 mg charcoal/kg mouse (administered orally in 100 ul volume saline), based on initial dose titration studies in a model of endotoxemia and on the known natural history of CM in C57BL/6 mice. Mice were not anesthetized or sedated during dosing as this frequently resulted in airway contamination. All vehicle-treated controls developed severe neurological symptoms, including convulsions and ataxia from 5-6 days post-infection, and died rapidly thereafter. In contrast, mice treated with activated charcoal were highly resistant to the development of CM (with a day 7 survival rate of approximately 95% compared to approximately 20% in control mice; FIG. 9a). Only approximately 15% of the activated charcoal-treated animals developed any clinical evidence of CM. As no anti-malarial agents were administered, activated charcoal-treated mice eventually became hyper-parasitemic, and died presumably from anemia. Nevertheless, administration of activated charcoal significantly prolonged overall survival time (FIG. 9a x²=19.18, P<0.00001). Strikingly, some treated animals survived for long periods despite parasitemia in excess of 75% (FIG. 9b). To our knowledge there is no comparable treatment that confers this level of protection against CM and malaria-induced death.

To determine whether activated charcoal inhibited brain histopathology, brain sections were stained with hematoxylin and eosin, and examined using a Zeiss Axiophot microscope with an Optronics CCD camera. Compared to normal brain, brains from vehicle-treated infected mice showed evidence of intra-cerebral injury, including peri-vascular haemorrhages containing parasitised red blood cells. In addition, many blood vessels were extensively occluded with thrombi composed of parasitized erythrocytes. In contrast, these histological changes were not observed in mice treated with activated charcoal.

Our data indicate that oral administration of activated charcoal almost completely inhibits the clinical and histopathological signs of CM in mice. In those mice that do develop CM (approximately 15%), onset is delayed, and in surviving mice charcoal also appears to provide a degree of protection against death due to high parasitemia. Activated charcoal may provide a first line therapy in the immediate absence of alternate treatment. Severe malaria is an acute illness, with neurological symptoms occurring often within 96 hours of the onset of fever; much of this time may be spent traveling from remote villages to health clinics and consequently many children arrive in coma. Our study indicates that charcoal therapy alone given early in the course of infection can dramatically prolong survival and restrict the development of neurological sequelae. In addition, recent studies indicate that “adjunctive” therapy during the first 24 hours of hospitalization may significantly decrease mortality associated with severe malaria. Activated charcoal is also be highly beneficial in this context. Oral activated charcoal has other attributes. It has been used for many years in the treatment of poisoning, including incidentally quinine poisoning. It is well tolerated and has a well-documented safety profile, is relatively inexpensive and administration is not technically demanding. The long shelf life, particularly in powdered form, makes it highly suited for use in remote rural communities.

In conclusion, oral charcoal can be a readily-implemented therapy for the treatment of severe malaria.

### EXAMPLE 3

BALB/c mice were intra-gastrically gavaged with 200 μl activated Charcoal (400 mg/kg) or 200 μl non-pyrogenic saline. Mice were infected intranasally with 50 HA units of influenza virus X31 in 50 μl non-pyrogenic saline. Mice were monitored daily and weight loss measured throughout infection. Mice were killed 7 days post infection (corresponding to height of immunopathology) by the injection of 3 mg pentobarbitone and exsanguination of the femoral vessels.

Broncho-alveolar lavage (BAL) fluid, lung tissue, mediastinal lymph node, spleen and Peyer's patches were obtained from each mouse as described previously (Hussell, T et al. 1996. J. Gen. Virol. 77:2447-2455). In brief, lungs were inflated six times with 1.5 ml of Eagle's Minimum Essential Medium (Sigma) containing 10 mM EDTA and kept on ice (BAL fluid), centrifuged, the supernatant decanted and the cell pellet resuspended to 1×10⁶ cells/ml in RPMI containing 10 % FCS, 2 mM/ml L-glutamine, 50 μg/ml penicillin and 50 μg/ml streptomycin (R10F). Solid tissue was disrupted using 0.8 μm filters to obtain single cell suspensions, the red blood cells lysed and the cell pellet re-suspended at 1×10⁶ cells/ml in R10F. Cell number was quantified using a haemocytometer and trypan blue exclusion. A single lobe of lung tissue was fixed in 2% formaldehyde and embedded in paraffin. Sections were stained with H and E.

1×10⁶ cells obtained from the airways or the lung were stained with the following antibody combinations: 1) anti-CD45RB-FITC, anti-CD103-PE anti-CD4-PerCP and anti-CD8-APC 2) anti-Ly6G-FITC, anti-CD86-PE, anti-CD11b-PerCP and anti-CD11c-APC 3) anti-CD45RB-FITC, anti-FoxP3-PE, anti-CD4-PerCP and anti-CD8-APC 4) to detect intracellular cytokines 1×10⁶ cells were incubated with 50 ng/ml PMA (Sigma-Aldrich), 500 ng ionomycin (Calbiochem) and 10 μg/ml brefeldin A (Sigma) for 4 h at 37° C. Cells were stained with anti-CD4-PerCP and anti-CD8-APC on ice for 30 min, washed and then fixed in 2% formaldehyde for 20 min at room temperature. Cells were permeabilised with 0.5% saponin in PBS containing 1% BSA and 0.1% azide for 10 min. A combination of anti-TNF-α-FITC anti-IL-4-PE, diluted in saponin buffer, was then added to the cells. After 30 min cells were washed once in saponin buffer and twice in PBS containing 0.1% azide and 1% BSA. Samples were analysed on an LSR flow cytometer (BD Biosciences), collecting data on at least 30,000 lymphocytes.

The cell numbers in the BAL and lung tissue of the influenza infected mice, treated with saline and charcoal, are illustrated in FIGS. 10 and 11. There is a substantially lower number of cells in the BAL of the charcoal treated mice in comparison with the saline treated mice (FIG. 11).

Influenza induces infiltration of inflammatory cells into the lungs causing inflammation. These results clearly demonstrate that charcoal suppresses infiltration of cells into the airways, which suppresses inflammation in the lungs.

### EXAMPLE 4

Starch elicited macrophages were obtained from DBA/1 mice by the intra peritoneal injection of a freshly prepared 1% starch solution. The mice were orally gavaged with either saline or charcoal (400 mg/kg) on day 1 and day 3 during the four day period. Macrophages were obtained as the plastic adherent cells from peritoneal exudates population and grown in culture in the presence or absence of LPS (10 ng/ml). Tumor necrosis factor was assayed from the culture supernatants harvested 24 h later by a sandwich ELISA.

The results are shown in FIG. 12. The results shows that oral gavaging with charcoal diminishes LPS (lipopolysaccharide) induced TNFα release from starch elicited peritoneal macrophages.

### EXAMPLE 5

An air pouch model of inflammatory mediator accumulation and cellular recruitment was set up in DBA/1 mice. Mice were orally gavaged with either saline or charcoal (400 mg/kg), two hours post per os treatment the air pouches on these mice were challenged with zymosan and four hours later the cellular pouch exudates were harvested and viable cell counts were taken from each mouse. The results are shown in FIG. 13. The results show that oral gavaging with activated charcoal reduces cell ingress at the site of inflammation.

### EXAMPLE 6

BALB/c mice were intra-gastrically gavaged with 100 μl activated charcoal (400 mg/kg) or 100 μl non-pyrogenic saline at day −1 and/or day 2.

Mice were infected intranasally with 50 HA units of influenza virus X31 in 50 μl non-pyrogenic saline at day 0. Mice were monitored daily and weight loss measured throughout infection. Mice were killed 6/7 days post infection (corresponding to height of immunopathology) by the injection of 3 mg per pentobarbitone and exsanguination of the femoral vessels.

Broncho-alveolar lavage (BAL) fluid and lung tissue were obtained from each mouse as described previously (Hussell, T et al 1996. J. Gen. Virol. 77:2447-2455). In brief, lungs were inflated six times with 1.5 ml of Eagle's Minimum Essential Medium (Sigma) containing 10 mM EDTA and kept on ice (BAL fluid), centrifuged, the supernatant collected to assay for cytokines by ELISA and the cell pellet re-suspended for counting. Solid tissue was disrupted using 0.8 μm filters to obtain single cell suspensions, the red blood cells lysed and the cell pellet re-suspended for counting.

Cell number was quantified using a haemocytometer and trypan blue exclusion. Cytokines in the BAL fluid were assayed by sandwich ELISA.

The results are shown in FIGS. 14 and 15a, b and c.

The conclusions from this work are:
Charcoal given at either 1 day prior to or 2 days after the Influenza infection reduces WBC trafficking to the lung (site of inflammation).
Mice given charcoal before infection seem to lose less body weight after infection.

### EXAMPLE 7

The most widely studied animal model of severe sepsis is lethal polymicrobial peritonitis caused by a surgical procedure termed “cecal ligation and puncture” (CLP). Here, mice were subjected to CLP, and treated orally with clinically achievable doeses of activated charcoal. Survival increased from 30% in vehicle-treated controls to 80% in activated charcoal-treated mice (FIG. 16d). Animals were followed for three weeks after the onset of sepsis, and no late deaths were observed, indicating that orally administered charcoal confers lasting protection, and does not merely delay death.

### Material and Methods

### Animals

Mice were 6-8 week old BALB/c or C57BL/6 mice (20-25 g) purchased from Harlan-Sprague-Dawley and allowed to acclimate for 7 days. Rats were adult males (280-300 g) from Charles River Laboratories. Both species were housed at 25° C. on a 12 hours light/dark cycle and allowed free access to water and their appropriate food.

### Endotoxemia

Mice were injected intraperitoneally with 7.5 mg endotoxin (Eschericia coli LPS 0111:B4; Sigma) that was dissolved in sterile, pyrogen-free saline at 5 mg/ml concentration and sonicated from 30 mins before each use. For the TNF blood determinations, the mice were killed at either 3 or 5 hours after LPS injection. Blood was collected from the heart, allowed to clot for 2 hours at room temperature and centrifuged for 20 mins at 1,500×g. Serum samples were stored at 20° C. before analysis. For the survival experiments, the mice were returned to their cages and observed till death or for two weeks. Blood was collected at different times after LPS administration, allowed to clot for 2 hours at room temperature, and centrifuged for 20 mins at 1,500×g.

### Sepsis

To induce a correlation of clinical bacteremia and sepsis, peritonitis was created in mice by the method of ceal ligation and puncture first described by Wichman et al. The animals were anesthetized with ketamine (100 mg/kg, i.m.) and xylazine (10 mg/kg, i.m.) and laparotomized. The cecum was ligated at the junction of ileocecal valve and the distal part punctured once with a 22-guage needle. Through this opening, a 1 mm length of stool was expressed and allowed to fall into the peritoneal cavity. The cecum was returned to its proper location and the abdomen was closed. After surgery each mouse was given an antibiotic (primazin; 0.5 mg/kg s.c) and 20 ml/kg of normal saline s.c. The mice were observed for three weeks.

### Oral Administration of Charcoal

Actidose-Aqua activated charcoal (0.2 g charcoal/ml) was obtained from Paddock laboratories, Inc. (Cat# NDC0574-0121-04). A range of concentration was first analyzed in endotoxemia to determine survival rate in a concentration dependent-fashion. Charcoal concentration range was obtained in water after a serial dilution from the original solution as follow; ¼ (50 mg charcoal/ml); ½ (25 mg charcoal/ml) and ¼ (6.25 mg charcoal/ml). Mice (25 g) were given a 100 μl of the solutions providing a final range of concentrations of 200, 100 and 25 mg charcoal/kg mouse. Mice were not anesthetized or sedated because mice with altered sensorial frequently resulted in airway contamination. In all experiments charcoal or water was administered 30 mins before endotoxin injection. Charcoal (100 mg Charcoal/kg mouse) was also analyzed in sepsis induced by CLP. Mice were also subjected to (100 or 5 mg Charcoal/kg mouse) to analyze the production of cytokines in the serum.

The results are shown in FIGS. 16 (a, b, c and d), which show that oral charcoal reduces serum cytokines and protects against lethal endotoxemia and sepsis. Details of FIGS. 16 (a, b, c and d) are as follows:

a Lewis rats (n=5) received endotoxin (15 mg/kg, i.v.l), and vehicle (o) or 25(▴) or 100 (♦) mg/kg activated charcoal by oral gavage immediately thereafter. Animals were euthanized at the times indicated and serum TNF measured by ELISA. ** P<0.05 for both charcoal-treated groups compared to control mice. B BALB/c mice (n=30) received endotoxin (7.5 mg/kg, i.p.) and control vehicle (o) or 25 (X), 100 (▴) or 200 (♦) mg/kg activated charcoal by oral gavage. Survival was monitored over 120 hours. **P<0.05 for groups treated with 100 or 200 mg/kg activated charcoal compared to control. c Control untreated mice or mice receiving 15 mg/kg endotoxin followed by vehicle (LPS), or followed by activated charcoal (LPS+Ch) were bled at 30 hours and serum HMGB1 was measured by quantitative immuno-blot as described previously. d Mice were subjected to cecal ligation and puncture (n=20 per group) and received either control vehicle (o) or 100 mg/kg activated charcoal (♦) by oral gavage. Survival was monitored over 120 hours. ** P<0.05, two-tailed Logrank Test.

1. Use of charcoal in the manufacture of an oral composition for the treatment of an inflammatory condition other than an inflammatory bowel disease and other than interstitial or other inflammation within the kidney.

2. The use according to claim 1, wherein the charcoal is activated charcoal.

3. The use according to claim 1, wherein the anti -inflammatory condition is one or more of: an autoimmune inflammatory conditions, a malarial inflammatory condition, inflammation associated with cancer, lung associated inflammatory disease, infection associated inflammation and injury associated inflammation.

4. The use according to claim 1, wherein the oral composition also comprises a further anti-inflammatory agent.

5. The use according to claim 4, wherein the further anti-inflammatory agent is a non-steroidal anti-inflammatory agent (NSAID) a disease modifying anti-rheumatic drug (DMARD), a biological agent, a steroid, an immunosuppressive agent, a salicylate and/or a microbicidal agent.

6. The use according to claim 1, for the treatment of a mammal, in particular a human.

7. A pharmaceutical composition comprising charcoal in combination with a further anti-inflammatory agent.

8. The composition according to claim 7, wherein the charcoal is activated charcoal.

9. The composition according to claim 7, wherein the further anti-inflammatory agent is a non-steroidal anti-inflammatory agent (NSAID), a disease modifying anti-rheumatic drug (DMARD), a biological agent, a steroid, an immunosuppressive agent, salicylate and/or a microbicidal agent.

10. A pharmaceutical composition, as claimed in claim 7, for use in the treatment of an inflammatory condition.

11. The composition according to claim 10, wherein the inflammatory condition is in a mammal.

12. The composition according to claim 11, wherein the mammal is a human.

13. A method of treating an inflammatory condition in a subject, other than an inflammatory bowel disease and other than interstitial or other inflammation within the kidney, comprising the oral administration of charcoal to the subject.

14. The method according to claim 13, wherein the charcoal is activated charcoal.

15. A method according to claim 13, wherein the charcoal is used in combination with a further inflammatory agent.

16. A method according to claim 13 wherein the further anti-inflammatory agent is a non-steroidal anti-inflammatory agent (NSAID), a disease modifying anti-rheumatic drug (DMARD), a biological agent, a steroid, an immunosuppressive agent, a salicylate and/or a microbicidal agent.

17. The method of claim 13, wherein the subject is a mammal.

18. The method of claim 17, wherein the mammal is a human.

19. The method of claim 13, wherein the inflammatory condition is one or more of an autoimmune inflammatory condition, a malarial inflammatory condition, inflammation associated with cancer, a lung associated inflammatory disease, infection associated inflammation and injury associated inflammation.
